(19) United States
(12) Patent Application Publication
(10) Pub. No.: US 2024/0416322 A1
(43) Pub. Date: Dec. 19, 2024

(54) CATALYSTS ON HIBONITE-TYPE SUPPORTS FOR CATALYTIC OXIDATIVE COUPLING OF METHANE

(71) Applicant: Lummus Technology LLC, Houston, TX (US)

(72) Inventors: Gary Podrebarac, Friendswood, TX (US); Zan Liu, Katy, TX (US); Khalid Azzam, Sugarland, TX (US); Daniel Rosenberg, Dunaujvaros (HU)

(21) Appl. No.: 18/673,834

(22) Filed: May 24, 2024

Related U.S. Application Data
(60) Provisional application No. 63/504,577, filed on May 26, 2023.

(51) Int. Cl. B01J 23/10; B01J 21/04; B01J 23/02; B01J 23/42; B01J 35/00; B01J 35/50

(52) U.S. Cl. CPC B01J 23/10; B01J 21/04; B01J 23/02; B01J 23/42; B01J 35/19; B01J 35/50

(57) ABSTRACT

Catalytic material having catalysts supported by hibonite-type supports are provided. The catalytic materials include a first catalytic material comprising an oxidative coupling methane catalyst for oxidative coupling of methane (OCM) and a second selective oxidation catalytic material comprising a selective oxidation catalyst that preferentially oxidizes hydrogen and carbon monoxide over methane. Systems comprising the first and second catalytic materials for performing an OCM reaction using a low temperature feedstock gas mixture and methods of using the same to prepare C₂₊ compounds are also provided.

### CROSS-REFERENCE TO RELATED APPLICATION(S)

This application is a continuation of U.S. Patent Application No. 63/504,577, filed May 26, 2023, which is hereby incorporated by reference as if fully set forth herein.

### BACKGROUND

### Technical Field

The present disclosure is generally related to supported catalysts and, more specifically, to catalysts on hibonite-type supports useful in a variety of catalytic reactions, such as the oxidative coupling of methane to produce C₂₊ hydrocarbons.

### Description of the Related Art

Catalysis is the process in which the rate of a chemical reaction is either increased or decreased by means of a catalyst. Positive catalysts lower the rate-limiting free energy change to the transition state, and thus increase the speed of a chemical reaction at a given temperature. Negative catalysts have the opposite effect. Substances that increase the activity of a catalyst are referred to as promoters or activators, and substances that deactivate a catalyst are referred to as catalytic poisons or deactivators. Unlike other reagents, a catalyst is not consumed by the chemical reaction, but instead participates in multiple chemical transformations. In the case of positive catalysts, the catalytic reaction generally has a lower rate-limiting free energy change to the transition state than the corresponding uncatalyzed reaction, resulting in an increased reaction rate at the same temperature. Thus, at a given temperature, a positive catalyst tends to increase the yield of desired product while decreasing the yield of undesired side products. Although catalysts are not consumed by the reaction itself, they may be inhibited, deactivated or destroyed by secondary processes, resulting in loss of catalytic activity.

Catalysts are generally characterized as either heterogeneous or homogeneous. Heterogeneous catalysts exist in a different phase than the reactants (e.g., a solid metal catalyst and gas phase reactants), and the catalytic reaction generally occurs on the surface of the heterogeneous catalyst. Thus, for the catalytic reaction to occur, the reactants must diffuse to and/or adsorb onto the catalyst surface. This transport and adsorption of reactants are often the rate limiting steps in a heterogeneous catalysis reaction. Heterogeneous catalysts are also generally easily separable from the reaction mixture by common techniques such as filtration or distillation.

One heterogeneous catalytic reaction with commercial potential is the oxidative coupling of methane (OCM) to ethylene: 2CH₄₊O₂→C₂H₄₊2H₂O. See, e.g., Zhang, Q., Journal of Natural Gas Chem., 12:81, 2003; Olah, G. “Hydrocarbon Chemistry”, Ed. 2, John Wiley & Sons (2003). This reaction is exothermic (ΔH=−67 kcals/mole) and has typically been shown to occur at very high temperatures (>700° C.). The OCM process includes both a heterogenous catalytic step, which involves the activation of oxygen and methane on the catalyst surface to generate methyl radicals (CH₃.), and a homogeneous gas-phase step, involving the coupling of methyl radicals to ethane (C₂H₆) followed by dehydrogenation to ethylene (C₂H₄). Activating O₂ into desirable species on the catalyst surface is a pivotal step that governs the methane activation to methyl radicals and subsequent oxidative dehydrogenation of ethane.

The use of heterogeneous catalysts, for example in the OCM reaction, presents a number of challenges, especially on a commercial scale. Commercial catalytic processes must be able to achieve a high conversion of the reactant (e.g., hydrocarbon) feedstock at high gas hourly space velocities. However, when a fixed bed of heterogeneous catalyst is used, the pressure drop across the catalyst bed prevents operation under the high gas space velocities demanded of a commercial operation. In addition, many commercially important catalytic reactions, such as OCM, are exothermic and controlling the exotherm (i.e., hotspots) within the catalyst bed can be difficult. Finally, many commercially important heterogeneous catalysts contain expensive and/or rare metals, so methods to reduce the amount of catalyst used for a given process are generally desirable.

To address these challenges, many heterogeneous catalysts are employed in combination with a support. The use of the support provides certain advantages. For example, the support provides a surface on which the catalyst is spread, increasing the effective surface area of the catalyst and reducing the required catalyst load. The support may also interact synergistically with the catalyst to enhance the catalytic properties of the catalyst.

The OCM process occurs under conditions of high temperature (typically between 650° C. and 950° C.), high pressure and/or high water vapor pressure due to the high bond strength (bond dissociation energy) of the tetrahedral C—H bonds in methane. The prolonged exposure to high temperature, combined with a significant amount of oxygen and sometimes steam can result in catalyst deactivation by support sintering. Therefore, there is a continuing need for highly active and robust supported OCM catalysts capable of controlling exotherms and improving yield and selectivity in the oxidative coupling of methane.

### BRIEF SUMMARY

In brief, catalytic materials comprising catalysts supported on hibonite-type supports for oxidative coupling of methane (OCM), catalytic materials comprising catalysts supported on hibonite-type supports for selective oxidation of hydrogen and/or carbon monoxide over methane, and OCM reactors and methods of using such catalytic materials are disclosed.

In one aspect, provided herein is a catalytic material for oxidative coupling of methane (OCM) comprising a support comprising an alkaline earth metal hexaaluminate and an OCM catalyst in contact with the support. The OCM catalyst comprises a rare earth oxide and at least one dopant.

A method for use of such catalytic material in the OCM reaction is also provided.

In one aspect, provided herein is a catalytic material for selective oxidation of at least hydrogen over methane comprising a support comprising an alkaline earth metal hexaaluminate and a selective oxidation catalyst (SOC) in contact with the support. The SOC comprises a noble metal.

A method for use of such catalytic material in the catalytic selective oxidation reaction is also provided.

In one aspect, provided herein is method for oxidative coupling of methane (OCM). The method comprises introducing a gas mixture feed comprising methane, oxygen and hydrogen to a reactor, wherein the reactor comprises a selective oxidation catalytic material and an OCM catalytic material; contacting the gas mixture feed with the selective oxidation catalytic material to combust hydrogen in the gas mixture feed and generate a heated gas mixture having a temperature capable of initiating an OCM reaction, the selective oxidation catalytic material comprising a selective oxidation catalyst (SOC) in contact with a first support; and contacting the heated gas mixture with the OCM catalytic material to initiate an OCM reaction and produce an OCM effluent comprising C₂₊ compounds including ethylene, the OCM catalytic material comprising an OCM catalyst in contact with a second support. At least one of the first support and the second support comprises an alkaline earth metal hexaaluminate.

These and other aspects of the invention will be apparent upon reference to the following detailed description. To this end, various references are set forth herein which describe in more detail certain background information, procedures, compounds and/or compositions, and are each hereby incorporated by reference in their entirety.

### BRIEF DESCRIPTION OF THE SEVERAL VIEWS OF THE DRAWINGS

In the drawings, the sizes and relative positions of elements in the drawings are not necessarily drawn to scale. For example, the various elements and angles are not drawn to scale, and some of these elements are arbitrarily enlarged and positioned to improve drawing legibility. Further, the particular shapes of the elements as drawn are not intended to convey any information regarding the actual shape of the particular elements, and have been selected solely for ease of recognition in the drawings.

FIGS. 1A-1D illustrate top views of exemplary support shapes, in accordance with embodiments of the present disclosure.

FIG. 2A is a side view depiction of a support, in accordance with embodiments of the present disclosure.

FIG. 2B is an end view showing the top of the support, in accordance with embodiments of the present disclosure.

FIG. 3 schematically depicts an oxidative coupling of methane reaction at the surface of a metal oxide catalyst.

FIG. 4 illustrates an exemplary system for oxidative coupling of methane, in accordance with embodiments of the present disclosure.

FIG. 5 illustrates an exemplary system for oxidative coupling of methane, in accordance with embodiments of the present disclosure.

FIG. 6 illustrates an exemplary system for oxidative coupling of methane, in accordance with embodiments of the present disclosure.

FIG. 7 shows comparative hydrothermal stability results for a hibonite-supported catalyst and an alpha-alumina supported catalyst in oxidative coupling of methane.

FIG. 8 shows combustion selectivity of a hibonite supported platinum catalyst in a selective oxidation reaction, in accordance with embodiments of the present disclosure.

FIG. 9 shows combustion selectivity of a comparative gamma-alumina supported platinum catalyst in a selective oxidation reaction, in accordance with embodiments of the present disclosure.

### DETAILED DESCRIPTION OF THE INVENTION

In the following description, certain specific details are set forth in order to provide a thorough understanding of various embodiments. However, one skilled in the art will understand that the invention may be practiced without these details. In other instances, well-known structures have not been shown or described in detail to avoid unnecessarily obscuring descriptions of the embodiments. Unless the context requires otherwise, throughout the specification and claims which follow, the word “comprise” and variations thereof, such as, “comprises” and “comprising” are to be construed in an open, inclusive sense, that is, as “including, but not limited to.” Further, headings provided herein are for convenience only and do not interpret the scope or meaning of the claimed invention.

Reference throughout this specification to “one embodiment” or “an embodiment” means that a particular feature, structure or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments. Also, as used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

### Definitions

As used herein, and unless the context dictates otherwise, the following terms have the meanings as specified below.

“Catalyst” means a substance which alters the rate of a chemical reaction. A catalyst may either increase the chemical reaction rate (i.e. a “positive catalyst”) or decrease the reaction rate (i.e. a “negative catalyst”). Catalysts participate in a reaction in a cyclic fashion such that the catalyst is cyclically regenerated. “Catalytic” means having the properties of a catalyst.

“Catalytic material” refers to a plurality of catalyst particles in combination with a support.

An “extrudate” refers to a material prepared by forcing such material in a semisolid state through a die or opening of appropriate shape. Extrudates can be prepared in a variety of shapes and structures by common means known in the art.

A “pellet” or “pressed pellet” refers to a material prepared by applying pressure to (i.e., compressing) the material into a desired shape. Pellets having various dimensions and shapes can be prepared according to common techniques in the art.

“Monolith” or “monolith support” is generally a structure formed from a single structural unit preferably having passages disposed through it in either an irregular or regular pattern with porous or non-porous walls separating adjacent passages. Examples of such monolithic supports include, e.g., ceramic or metal foam-like or porous structures. The single structural unit may be used in place of or in addition to conventional particulate or granular forms (e.g., pellets or extrudates). Monoliths generally have a porous fraction ranging from about 60% to 90% and a flow resistance substantially less than the flow resistance of a packed bed of similar volume (e.g., about 10% to 30% of the flow resistance of a packed bed of similar volume).

“Active” or “catalytically active” refers to a catalyst which has substantial activity in the reaction of interest. For example, in some embodiments a catalyst which is OCM active (i.e., has activity in the OCM reaction) has a C2 selectivity of 5% or more and/or a methane conversion of 5% or more when the catalyst is employed as a heterogenous catalyst in the oxidative coupling of methane at a temperature of 950° C. or less.

“Light-off temperature” is the temperature at which a catalyst or catalytic material has sufficient catalytic activity to initiate the desired reaction. In certain embodiments, e.g., for exothermic reactions like OCM, the light-off temperature is at a sufficient level to not only allow initiation of the catalyzed reaction, but to do so at a rate that is thermally self-sufficient, e.g., generating enough thermal energy to maintain the reaction temperature at or above the initiation temperature.

“Dopant” is chemical compound which is added to or incorporated within a catalyst base material to optimize catalytic performance (e.g. increase or decrease catalytic activity). As compared to the undoped catalyst, a doped catalyst may increase or decrease the selectivity, conversion, and/or yield of a reaction catalyzed by the catalyst. Dopants which increase catalytic activity are referred to as “promoters” while dopants which decrease catalytic activity are referred to as “poisons”. The dopant may be present in the catalyst in any form and may be derived from any suitable source of the element (e.g., chlorides, bromides, iodides, nitrates, oxynitrates, oxyhalides, acetates, formates, hydroxides, carbonates, phosphates, sulfates, alkoxides, and the like.)

“Atomic percent” (at % or at/at) or “atomic ratio” when used in the context of catalyst dopants refers to the ratio of the total number of dopant atoms to the total number of non-oxygen atoms in the base material. For example, the atomic percent of dopant in a strontium doped La₂O₃ catalyst is determined by calculating the total number of strontium atoms and dividing by the sum of the total number of lanthanum atoms and multiplying by 100 (i.e., atomic percent of dopant=[Sr atoms/La atoms)]×100).

“Metal salt” includes metal acids and salts of metal acids.

“Conversion” means the mole fraction (i.e., percent) of a reactant converted to a product or products.

“Selectivity” refers to the percent of converted reactant that went to a specified product, e.g., C2 selectivity is the % of converted methane that formed ethane and ethylene, C₃ selectivity is the % of converted methane that formed propane and propylene, CO selectivity is the % of converted methane that formed CO. C₂₊ selectivity is the sum of the C₂ selectivity and C₃ selectivity.

“Yield” is a measure of (e.g. percent) of product obtained relative to the theoretical maximum product obtainable. Yield is calculated by dividing the amount of the obtained product in moles by the theoretical yield in moles. Percent yield is calculated by multiplying this value by 100. C₂ yield is defined as the sum of the ethane and ethylene molar flow at the reactor outlet multiplied by two and divided by the inlet methane molar flow. C₃ yield is defined as the sum of propane and propylene molar flow at the reactor outlet multiplied by three and divided by the inlet methane molar flow. C₂₊ yield is the sum of the C₂ yield and C₃ yield. Yield is also calculable by multiplying the methane conversion by the relevant selectivity, e.g. C₂ yield is equal to the methane conversion times the C₂ selectivity.

“C₂” yield is the total combined yield of ethane and ethylene.

“C₂” selectivity is the combined selectivity for ethane and ethylene.

“Oxide” refers to a metal compound comprising oxygen. Examples of oxides include, but are not limited to, metal oxides (MₓO_{y}), metal oxyhalide (MₓO_{y}X_{z}), metal oxynitrates (MₓO_{y}(NO₃)ₓ), metal phosphates (Mₓ(PO₄)_{y}), and the like, wherein x, y and z are numbers from 1 to 100.

“Mixed oxide” or “mixed metal oxide” refers to a compound comprising two or more oxidized metals and oxygen (i.e., M1ₓM2_{y}O_{z}, wherein M1 and M2 are the same or different metal elements, O is oxygen and x, y and z are numbers from 1 to 100). A mixed oxide may comprise metal elements in various oxidation states and may comprise more than one type of metal element. Mixed oxides comprising 2, 3, 4, 5, 6 or more metal elements can be represented in an analogous manner. Mixed oxides also include oxy-hydroxides (e.g., MₓO_{y}OH_{z}, wherein M is a metal element, O is oxygen, x, y and z are numbers from 1 to 100 and OH is hydroxy). Mixed oxides may be represented herein as M1-M2, wherein M1 and M2 are each independently a metal element.

“Rare earth oxide” refers to an oxide of an element from group 3, lanthanides or actinides. Rare earth oxides include mixed oxide containing a rare earth element. Examples of rare earth oxides include, but are not limited to, La₂O₃, Nd₂O₃, Yb₂O₃, Eu₂O₃, Sm₂O₃, Y₂O₃, Ce₂O₃, CeO₂, Pr₂O₃, Ln1₄₋ₓLn2ₓO₆, La₄₋ₓLn1ₓO₆, La₄₋ₓNdₓO₆, wherein Ln1 and Ln2 are each independently a lanthanide element, wherein Ln1 and Ln2 are not the same and x is a number ranging from greater than 0 to less than 4, La₃NdO₆, LaNd₃O₆, La_{1.5}Nd_{2.5}O₆, La_{2.5}Nd_{1.5}O₆, La_{3.2}Nd_{0.8}O₆, La_{3.5}Nd_{0.5}O₆, La_{3.8}Nd_{0.2}O₆, Y—La, Zr—La, Pr—La and Ce—La.

“Catalyst precursor” refers to the catalyst support impregnated with a solution of the active metal salt, but prior to forming the metal oxide or metal element (by catalyst calcination). The catalyst precursor is a stable intermediate in the production of a supported catalyst.

### OCM Catalytic Materials

In one aspect, the present disclosure provides a catalytic material for oxidative coupling of methane (also referred to as “OCM catalytic material”). In some embodiments, the OCM catalytic material comprises one or more OCM catalysts in combination with a support.

The OCM catalyst may be any catalyst composition that is capable of facilitating an OCM reaction, such as the catalysts described in, for example, U.S. Pat. Nos. 8,921,256, 8,962,517, 9,738,571, 9,751,079, and 9,956,544 the full disclosures of which are incorporated herein by reference in their entirety.

In some embodiments, the OCM catalyst disclosed herein comprises a rare earth oxide (i.e., lanthanides, actinides and Group 3) and a dopant.

The rare earth oxide may comprise any rare earth element, and in some embodiments the rare earth element is a lanthanide element including lanthanum (La), cerium (Ce), praseodymium (Pr), neodymium (Nd), samarium (Sm), europium (Eu), gadolinium (Gd), ytterbium (Yb), or yttrium (Y).

In some embodiments, the rare earth oxide may comprise a lanthanide oxide including lanthanum oxide (La₂O₃), cerium (IV) oxide (CeO₂), cerium (III) oxide (Ce₂O₃), praseodymium oxide (Pr₂O₃), neodymium oxide (Nd₂O₃), samarium oxide (Sm₂O₃), europium oxide (Eu₂O₃), gadolinium oxide (Gd₂O₃), ytterbium oxide (Yb₂O₃), yttrium oxide (Y₂O₃), or combinations thereof.

In some embodiments, the rare earth oxide is a rare earth mixed oxide such as La₃NdO₆, LaNd₃O₆, La_{1.5}Nd_{2.5}O₆, La_{2.5}Nd_{1.5}O₆, La_{3.2}Nd_{0.8}O₆, La_{3.5}Nd_{0.5}O₆, La_{3.8}Nd_{0.2}O₆ or combinations thereof and the like. Certain lanthanide mixed oxides such as lanthanum yttrium oxide (LaYO₃), lanthanum zirconium oxide (La₂Zr₂O₇), or lanthanum-cerium (La₂Ce₂O₇) are also useful as OCM catalysts in the OCM reaction.

In some embodiments, the dopant is selected from alkaline earth metal elements. For example, in some embodiments the dopant is selected from beryllium (Be), magnesium (Mg), calcium (Ca), strontium (Sr), barium (Ba), and combinations thereof. In some more specific embodiments, the dopant is Be. In some more specific embodiments, the dopant is Ca. In some more specific embodiments, the dopant is Sr. In some more specific embodiments, the dopant is Ba. In some embodiments, the OCM catalyst may comprise Nd₂O₃ as a base material and Sr as a dopant. In some other embodiments, the OCM catalyst may comprise Nd₂O₃ as a base material and Mg as a dopant.

In some embodiments, the alkaline earth metal may be in the form of an oxide. For example, the dopant may be magnesium oxide (MgO), calcium oxide (CaO), strontium oxide (SrO), barium oxide (BaO), or combinations thereof. In some embodiments, the OCM catalyst may comprise Nd₂O₃ as a base material and SrO as a dopant. In some other embodiments, the OCM catalyst may comprise Nd₂O₃ as a base material and MgO as a dopant.

The rare earth oxide may be doped with varying amounts of alkaline earth metal elements to yield an alkaline earth metal doped rare earth oxide. In some embodiments, the dopant may be present in the OCM catalyst in up to 75% by weight of the OCM catalyst. For example, the rare earth oxide may be doped with about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 55% or 60% of an alkaline earth metal by weight of the OCM catalyst.

In other embodiments, the concentration of the dopant is measured in terms of atomic percent (at/at). In some of these embodiments, the dopant may be present in the OCM catalyst in up to 75% at/at. For example, in one embodiment the concentration of the alkaline earth metal element ranges from 0.01% to 1% at/at, 1%-5% at/at, 5%-10% at/at. 10%-20% at/at, 20%-30% at/at, 30%-40% at/at or 40%-50% at/at, for example about 1% at/at, about 2% at/at, about 3% at/at, about 4% at/at, about 5% at/at, about 6% at/at, about 7% at/at, about 8% at/at, about 9% at/at, about 10% at/at, about 11% at/at, about 12% at/at, about 13% at/at, about 14% at/at, about 15% at/at, about 16% at/at, about 17% at/at, about 18% at/at, about 19% at/at or about 20% at/at.

The OCM catalysts of the present disclosure may be analyzed by inductively coupled plasma mass spectrometry (ICP-MS) to determine the element content of the catalysts. ICP-MS is a type of mass spectrometry that is highly sensitive and capable of the determination of a range of metals and several non-metals at concentrations below one part in 10¹². ICP is based on coupling together an inductively coupled plasma as a method of producing ions (ionization) with a mass spectrometer as a method of separating and detecting the ions. ICP-MS methods are well known in the art.

The amount of the OCM catalyst in the OCM catalytic material can vary, for example, from 1 to 5 wt %, from 5 to 15 wt %, from 15 to 25 wt %, from 25 to 35 wt %, from 35 to 45 wt %, or from 45 to 55 w %. In some embodiments, the OCM catalyst is present in an amount about 5 wt %, about 10 wt %, about 12.5 wt %, about 15 wt %, about 17.5 wt %, or about 20 wt %.

The OCM catalyst is disposed on and/or impregnated in the support. In some embodiments, the OCM catalyst is impregnated in the support. In some embodiments, the support is a hibonite-type support. In some embodiments, the hibonite-type support comprises alkaline earth metal hexaaluminate such as calcium hexaaluminate (CaAl₁₂O₁₉), barium hexaaluminate (BaAl₁₂O₁₉), or strontium hexaaluminate (SrAl₁₂O₁₉).

In some embodiments, the support comprises hibonite (CaAl₁₂O₁₉), and the catalytic material comprises SrO doped Nd₂O₃ supported by the hibonite. In some other embodiments, the support comprises hibonite, and the catalytic material comprises MgO doped Nd₂O₃ supported by the hibonite.

The support in accordance with the present disclosure may be provided in any number of forms. In some embodiments, the support may be provided in the form of aggregated particles, for example, pellets or extrudates. In some other embodiments, the support may be provided in a monolithic form, e.g., blocks, honeycombs, foils, lattices, etc. In some embodiments, the support is provided as extruded or pelletized cylinders, spheres, rods, trilobes, tetralobes, rings, doughnuts, stars, cartwheels, or strands.

In some embodiments, the support has a non-tessellating shape. Non-tessellating shapes are advantageous in embodiments of the present disclosure since the resulting catalytic materials cannot tightly pack together and void spaces remains between the individual formed pieces. For example, the non-tessellating shape may be a circle, ellipse, or polygon (either regular or irregular) in top view.

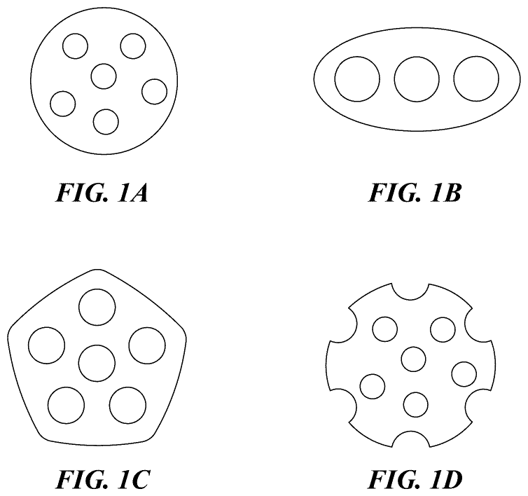

In some embodiments, the non-tessellating shape is a circle (e.g., FIG. 1A). In some embodiments, the circle has a diameter ranging from about 5 mm to 30 mm, for example from about 5 mm to about 10 mm, about 10 mm to about 20 mm, or about 20 mm to about 30 mm. In some more specific embodiments, the circle diameter is 5 mm, 8 mm, 10 mm, 12 mm, 14 mm, 16 mm, 18 mm, 20 mm, 22 mm, 22 mm, or 26 mm.

In other embodiments, the non-tessellating shape is an ellipse (e.g., FIG. 1B). In some embodiments, the ellipse has a major diameter ranging from about 10 mm to about 30 mm and a minor diameter ranging from about 5 mm to about 20 mm. In other embodiments, the ellipse has a major diameter ranging from about 20 mm to about 30 mm and a minor diameter ranges from about 6 mm to about 18 mm. For example, in some more specific embodiments, the major diameter ranges from about 22 mm to about 26 mm and the minor diameter ranges from about 10 mm to about 14 mm.

In some embodiments, the non-tessellating shape is a polygon (e.g., FIG. 1C). The polygon may be an irregular or a regular polygon. In some embodiments, the polygon has an effective diameter ranging from about 5 mm to about 50 mm, for example from about 5 mm to about 10 mm, about 10 mm to about 20 mm, from about 20 mm to about 30 mm, from about 30 mm to about 40 mm, or from about 40 mm to about 50 mm. In some more specific embodiments, the effective diameter ranges from about 22 to 26 mm. In some embodiments, the polygon is pentagon or hexagon.

In some embodiments, the support comprises rounded or chamfered edges. In some embodiments, the support includes grooves and/or flutes on the edges (e.g., FIG. 1D). The flutes are typically a convex shape (circular) cut into the outer edge of the support. In some embodiments, the support comprises one or more fluted edge. In some other embodiments, the one or more flutes have a diameter ranging from about 2 mm to about 10 mm, for example about 6 mm.

Further, since pressure drop across a catalyst bed is an important factor to consider, in certain embodiments, the support comprises convex surfaces, instead of the traditional flat surfaces. The convex surfaces allow for more void volume in the packed catalyst bed (i.e., the formed catalytic materials do not pack as tightly).

The support may include one or more holes extending therethrough. In some embodiments, the support has 1 to 12, 2 to 10, 2 to 6, 3 to 10, 3 to 6, 4 or 10, or 4-6 holes extending therethrough. In some embodiments, the holes may be equally spaced and symmetrically positioned about the cross section of the support so as to maximize the strength of the resulting catalytic material. Thus one hole may be centrally positioned, 3 holes may be in a triangular patter, 4 holes may be in a square pattern, 5 holes may be in a square pattern with a central hole, 6 holes may be in a hexagon pattern or a pentagon pattern with a central hole, and so on.

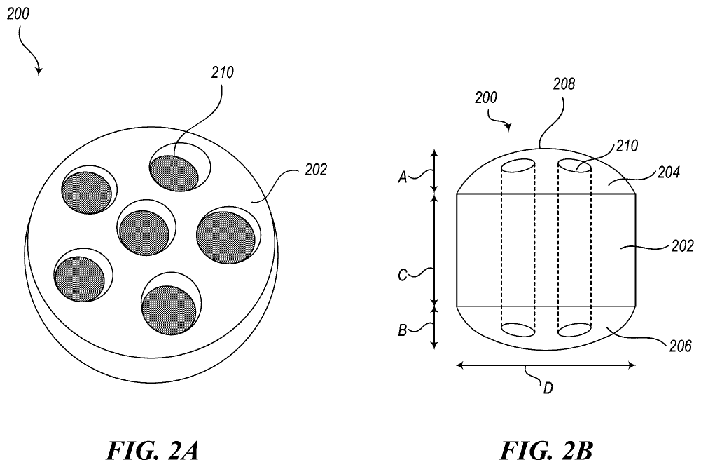

FIGS. 2A and 2B illustrates a support 200 that can be used in the present disclosure. Referring to FIGS. 2A and 2B, the support 200 is in the form of a cylinder 202 having a height C and diameter D. One or both ends 204, 206 of the cylinder 202 are domed. In some embodiments and as shown in FIG. 2B, for support 200, both ends 204, 206 of the cylinder 202 are domed with convex surfaces 208. The domed ends 204, 206 have heights A and B, which may be the same or different. In some embodiments, the heights A and B are the same.

The aspect ratio of the support 200, defined as overall heights divided by the diameter, i.e. (A+B+C)/D, may be set to be in the range from 0.5 to 2.0 or from 0.75 to 1.50 to reduce the tendency of the support pellets to stack while at the same time providing a reduced tendency to break.

In some embodiments, the ratio of the domed ends 204, 206 to the cylindrical part of the support 200 (i.e., (A+B)/C) may be set to be in the range from 0.4 to 3.0 or from 0.5 to 2.5. The domed ends 204, 206 may form a segment of a circle or ellipse in cross section, and desirably have a radium R≥D/2.

In some embodiments, the cylinder 202 has a height C in the range of 1 mm to 25 mm and a diameter D in the range of 4 mm to 40 mm.

In some embodiments, the support 200 has five symmetrically positioned holes 210 extending therethrough. The holes 210 have a circular shape in cross section.

The support comprises a BET (Brunauer, Emmett, Teller) surface area of from 0.1 and 100 m²/g, from 1 and 100 m²/g, from 1 and 50 m²/g, from 1 and 20 m²/g, from 1 and 10 m²/g, from 1 and 5 m²/g, from 1 and 4 m²/g, from 1 and 3 m²/g, or from 1 and 2 m²/g, as measured by nitrogen absorption. In some embodiments, the support has a BET surface area of 20 to 80 m²/g. In some embodiments, at least 50%, 60%, 70%, 80%, 90%, 95%, or 100% the surface area of the support can be in direct contact (e.g., coated and/or impregnated with) with the OCM catalyst.

The support comprises a pore volume of greater than about 0.1 cubic centimeters per gram (cc/g), greater than about 0.2 cc/g, greater than about 0.3 cc/g, greater than about 0.4 cc/g, greater than about 0.5 cc/g, greater than about 0.6 cc/g, greater than about 0.7 cc/g, greater than about 0.8 cc/g, greater than about 0.9 cc/g or greater than about 1.0 cc/g, as determined by mercury intrusion porosimetry. In some embodiments, the support comprises a pore volume of 0.2 to 0.65 cm³/g.

In some more specific embodiments, the support has a porosity between 40% and 60%, a pore volume between 0.2 cc/g and 0.65 cc/g, and a BET surface area between 20 and 80 m²/g.

The foregoing catalytic materials disclosed in various embodiments herein, when used as a heterogeneous catalyst in the oxidative coupling of methane, the OCM catalytic material is capable of converting methane into C₂₊ hydrocarbons with a C₂₊ selectivity of at least 10%, at least 20%, at least 30%, at least 35%, at least 40%, at least 45%, at least 50%, at least 55%, at least 60%, at least 65%, or at least 70% at a temperature of at least 100° C., at least 200° C., at least 300° C., at least 400° C., at least 450° C., at least 480° C., at least 490° C., at least 500° C., at least 510° C., at least 520° C., at least 550° C., at least 600° C., at least 650° C., at least 700° C., at least 750° C., at least 800° C., at least 850° C., at least 900° C. or at least 950° C. In some embodiments, the OCM catalyst is converting methane into C₂₊ hydrocarbons with a C₂₊ selectivity of 35% to 85% including a C₂₊ selectivity of 40% to 85%, 50% to 85%, 60% to 85%, and also including a C₂₊ selectivity of 70% to 85%.

The foregoing catalytic materials disclosed in various embodiments herein, when used as a heterogeneous catalyst in the oxidative coupling of methane, the OCM catalytic material is capable of converting methane into C₂₊ hydrocarbons with a methane conversion at least 5%, at least 8%, at least 10%, at least 12%, at least 14%, at least 15%, at least 18%, at least 20%, at least 22% or at least 25% at a temperature of at least 100° C., at least 200° C., at least 300° C., at least 400° C., at least 450° C., at least 480° C., at least 490° C., at least 500° C., at least 510° C., at least 520° C., at least 550° C., at least 600° C., at least 650° C., at least 700° C., at least 750° C., at least 800° C., at least 850° C., at least 900° C. or at least 950° C.

The foregoing catalytic materials disclosed in various embodiments herein, when used as a heterogeneous catalyst in the oxidative coupling of methane, the OCM catalytic material is capable of converting methane into C₂₊ hydrocarbons with a yield of at least 10%, at least 20%, at least 30%, at least 35%, at least 40%, at least 50%, at least 60%, at least 70%, at least 80% or at least 90% at an inlet temperature of at least 100° C., at least 200° C., at least 300° C., at least 400° C., at least 450° C., at least 480° C., at least 490° C., at least 500° C., at least 510° C., at least 520° C., at least 550° C., at least 600° C., at least 650° C., at least 700° C., at least 750° C., at least 800° C., at least 850° C. or at least 900° C.

The foregoing catalytic materials disclosed in various embodiments herein, when used as a heterogeneous catalyst in the oxidative coupling of methane, the OCM catalytic material is capable of reaching a C₂₊ selectivity of at least 10%, at least 20%, at least 30%, at least 40%, at least 50%, at least 60%, at least 70%, at least 80% or at least 90% at a pressure at least above 1 barg, above at least about 2 barg, above at least about 3 barg, above at least about 4 barg, above at least about 5 barg, above at least about 6 barg, above at least about 8 barg or above at least about 10 barg.

In some more specific embodiments, the OCM catalytic material comprises a C₂₊ selectivity of at least 20% when the catalyst is employed as a heterogeneous catalyst in oxidative coupling of methane at a temperature of at least 400° C. and a pressure of at least about 2 barg.

In other more specific embodiments, the OCM catalytic material comprises the catalyst comprises a C₂₊ selectivity of at least 20% when the catalyst is employed as a heterogeneous catalyst in the OCM at a temperature of at least 600° C. and a pressure of at least about 8 barg.

The OCM catalytic materials disclosed in various embodiments herein are stable after a given time of operation under an OCM reaction temperature. In some embodiments, the OCM catalytic material can maintain at least 90% of the C₂₊ selectivity after being employed as a heterogeneous catalyst in the oxidative coupling of methane for at least about 1,000 hours, at least about 2,000 hours, at least about 5,000 hours, at least about 10,000 hours or at least about 20,000 hours. In some other embodiments, the OCM catalytic material can maintain at least 90% of the C₂₊ selectivity after the catalyst is employed as a heterogeneous catalyst in the oxidative coupling of methane for at least about 1,000 hours, at least about 2,000 hours, at least about 5,000 hours, at least about 10,000 hours or at least about 20,000 hours at gas hourly space velocity (GHSV).

### Preparation of OCM Catalytic Materials

In one aspect, the present disclosure provides a method for preparation of OCM catalytic materials described above.

In some embodiments, the OCM catalytic materials are prepared by mixing a base catalyst material precursor and a dopant precursor in a solvent to provide a catalyst precursor solution. Examples of solvents include, but are not limited to, water, methanol, ethanol, propanol, isopropanol, butanol, tetrahydrofuran, diethyl ether, dimethoxyethane, acetonitrile, toluene, or mixtures thereof. In some embodiments, the base catalyst material precursor includes a rare earth metal salt selected from a rare earth metal nitrate, a rare earth metal acetate, a rare earth metal citrate, a rare earth metal lactate, a rare earth metal carbonate, or combinations thereof. In some embodiments, the rare earth metal salt is lanthanum nitride or neodymium nitrate. In some embodiments, the dopant precursor includes an alkaline earth metal salt selected from an alkaline earth metal nitrate, an alkaline earth metal acetate, an alkaline earth metal citrate, an alkaline earth metal lactate, an alkaline earth metal carbonate, or combinations thereof. In some embodiments, the alkaline earth metal salt is strontium nitrate. In some embodiments, the catalyst precursor solution is prepared by dissolving neodymium (III) nitrate hexahydrate and strontium nitrate in DI water.

The catalyst precursor solution is applied to the support pellets to provide support pellets comprising the base catalyst precursor and the dopant precursor. In some embodiments, application of the catalyst precursor solution is performed by immersion of the support pellets in the catalyst precursor solution or by so-called “incipient wetness” impregnation where the volume of solution used equates approximately to the pore volume of the support pellets. Impregnation may be performed at ambient or elevated temperature and at atmospheric or elevated pressure. In some embodiments, the impregnation is performed at ambient temperature (i.e., 20 to 25° C.) and under atmospheric pressure (about 1 bar abs) for about one hour.

Following impregnation, the support pellets are removed from the catalyst precursor solution, and are then dried and calcined to provide the OCM catalytic materials of the present disclosure. The drying temperatures may be from 80 to 120° C. and drying times may be from 1 minutes to 6 hours. In some embodiments, the support pellets are dried in a convection oven at 110° C. for about four hours. The oven temperature is increased from the ambient temperature to 110° C. at a rate of 5° C./min. In some embodiments, the support pellets are dried in stagnant air with no gas flow. After drying, the oven temperature is increased from the drying temperature, e.g., 110° C., to a calcination temperature at a rate of 5° C./min. The calcination temperatures may be from 400 to 900° C., with calcination times ranging from 5 minutes to 5 hours. In some embodiments, the catalyst precursor-impregnated support pellets are calcinated at 800° C. for about 5 hours to decompose the metal salts into metal oxides. In some embodiments, the calcination decomposes the rare earth metal salt into the rare earth oxide, and the alkaline earth metal salt into the alkaline earth metal oxide.

After the calcination step, the support pellets comprising OCM catalyst are cooled from 800° C. to ambient temperature to afford the supported OCM catalyst.

During the heating steps, the cooling steps, and the steps of maintaining the temperature, the support pellets may be exposed to an atmosphere comprising oxygen. The atmosphere may comprise 15 mole % to 25 mole % oxygen and 75 mole % to 85 mole % nitrogen. In some embodiments, the atmosphere may be air.

The impregnation step and calcination step could be repeated one or more times in order to increase the amount of OCM catalyst on the support pellets.

### Systems and Methods for Oxidative Coupling of Methane Using OCM Catalytic Materials

In one aspect, the present disclosure provides OCM catalytic materials for improving the yield, selectivity and/or conversion of OCM reaction.

The selective, catalytic oxidative coupling of methane to ethylene (i.e. the OCM reaction) is shown by the following reaction (1):

2CH₄₊O₂→CH₂CH₂+2H₂O (1)

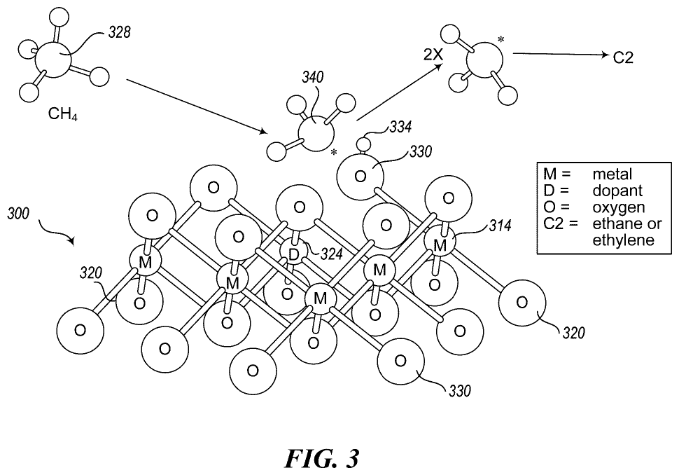

FIG. 3 shows schematically the first part of an OCM reaction that takes place on the surface of a metal oxide catalyst 300 which is followed by methyl radical coupling in the gas phase. A crystal lattice structure of metal atoms 314 and oxygen atoms 320 are shown, with a dopant 324 incorporated into the lattice structure. In this reaction, a methane molecule 328 comes into contact with an active site (e.g., surface oxygen 330) and becomes activated when a hydrogen atom 334 dissociates from the methane molecule 328. As a result, a methyl radical 340 is generated on or near the catalytic surface. Two methyl radicals thus generated can couple in the gas phase to create ethane and/or ethylene, which are collectively referred to as the “C₂” coupling products.

The yield of C₂H₄ and C₂H₆ is limited by further reactions in the gas phase and to some extent on the catalyst surface. A few of the possible reactions that occur during the oxidation of methane are shown below as reactions (2) through (8):

CH₄→CH₃ radical (2)

2 CH₃ radical→C₂H₆ (3)

CH₃ radical+1.7502→CO₂+1.5H₂O (4)

C₂H₆→C₂H₄+H₂ (5)

C₂H₆+0.5O₂→C₂H₄+H₂O (6)

C₂H₄+3O₂→2CO₂+2H₂O (7)

CH₃ radical+CₓH_{y}+O₂→Higher HC's-Oxidation/CO₂+H₂O (8)

The OCM catalytic materials of this disclosure are highly active and can optionally operate at a much lower temperature. In some embodiments, the OCM catalytic materials disclosed herein enable efficient conversion (i.e., high yield, conversion, and/or selectivity) of methane to ethylene at temperatures of less than 900° C., less than 800° C., less than 700° C., less than 600° C., less than 550° C., or less than 500° C. In other embodiments, the use of staged oxygen addition, designed heat management, rapid quench and/or advanced separations may also be employed.

Accordingly, one aspect of the present disclosure is a method for the preparation of ethane and/or ethylene, the method comprising converting methane to ethane and/or ethylene in the presence of an OCM catalytic material as disclosed herein. The OCM reaction is typically conducted by flowing a feed gas comprising a hydrocarbon or mixtures of hydrocarbons and oxygen through a catalyst bed comprising an OCM catalytic material.

In a preferred embodiment, the hydrocarbon is a mixture of hydrocarbons that is predominately methane (e.g., natural gas). The oxygen containing gas used in the present disclosure can be air, oxygen enriched air, or oxygen gas. The reactant gas mixture may further contain other gases, provided that these do not negatively affect the reaction. Examples of such other gases include carbon dioxide, nitrogen, helium, and hydrogen. The hydrogen may be from various sources, including streams coming from other chemical processes, like ethane cracking, methanol synthesis, or conversion of methane to aromatics. Carbon dioxide can be obtained from natural gas or form a waste or recycle gas stream.

The exothermic heats of reaction (free energy) follow the order of reactions depicted above and, because of the proximity of the active sites, will mechanistically favor ethylene formation while minimizing complete oxidation reactions that form CO and CO₂.

Important performance parameters used to measure the OCM catalytic materials' performance in the OCM reaction are selected from single pass methane conversion percentage (i.e., the percent of methane converted on a single pass over the catalytic material or catalytic bed, etc.), reaction inlet gas temperature, reaction operating temperature, total reaction pressure, methane partial pressure, gas-hour space velocity (GHSV), O₂ source, catalyst stability and ethylene to ethane ratio.

Typical temperatures for operating an OCM reaction according to the present disclosure are 950° C. or lower, 900° C. or lower, 850° C. or lower, 800° C. or lower, 750° C. or lower, 700° C. or lower, 650° C. or lower, 600° C. or lower 550° C. or lower, 500° C. or lower, 450° C. or lower, or 400° C. or lower. As used herein, the operation temperatures presented typically refer to the temperature immediately adjacent to the reactor inlet. As will be appreciated, with no integrated temperature control system, the exothermic nature of the OCM reaction can result in a temperature gradient across the reactor indicative of the progress of the reaction, where the inlet temperature can range from about 400° C. to about 600° C., while the outlet temperature ranges from about 700° C. to about 900° C. Typically, such temperature gradients can range from about 100° C. to about 500° C. By staging adiabatic reactors, with interstage cooling systems, one can step through a more complete catalytic reaction without generating extreme temperatures, e.g., in excess of 900° C.

In certain embodiments, the inlet gas temperature in an OCM reaction catalyzed by the disclosed OCM catalytic materials is <700° C., <675° C., <650° C., <625° C., <600° C., <575° C., <550° C., <525° C., <500° C., <490° C., <480° C., <470° C., <460° C., <450° C., <440° C., <430° C., or <420° C. In certain embodiments, the reaction operating temperature (i.e., outlet temperature) in an OCM reaction catalyzed by the disclosed OCM catalytic materials is <950° C., <925° C., <900° C., <875° C., <850° C., <825° C., <800° C., <775° C., <750° C., <725° C., <700° C., <675° C., <650° C., <625° C., <600° C., <590° C., <580° C., <570° C., <560° C., <550° C., <540° C., <530° C., <520° C., <510° C., <500° C., <490° C., <480° C., <460° C., or <450° C.

The single pass methane conversion in an OCM reaction catalyzed by the OCM catalytic materials is generally >5%, >10%, >15%, >20%, >25%, >30%, >35%, >40%, >45%, >50%, >55%, >60%, >65%, >70%, >75%, or even >80%.

In certain embodiments, the inlet reaction pressure in an OCM reaction catalyzed by the OCM catalytic materials is >1 atm, >1.1 atm, >1.2 atm, >1.3 atm, >1.4 atm, >1.5 atm, >1.6 atm, >1.7 atm, >1.8 atm, >1.9 atm, >2 atm, >2.1 atm, >2.1 atm, >2.2 atm, >2.3 atm, >2.4 atm, >2.5 atm, >2.6 atm, >2.7 atm, >2.8 atm, >2.9 atm, >3.0 atm, >3.5 atm, >4.0 atm, >4.5 atm, >5.0 atm, >5.5 atm, >6.0 atm, >6.5 atm, >7.0 atm, >7.5 atm, >8.0 atm, >8.5 atm, >9.0 atm, >10.0 atm, >11.0 atm, >12.0 atm, >13.0 atm, >14.0 atm, >15.0 atm, >16.0 atm, >17.0 atm, >18.0 atm, >19.0 atm, or >20.0 atm.

In some embodiments, the methane partial pressure in an OCM reaction catalyzed by the OCM catalytic materials is >0.3 atm, >0.4 atm, >0.5 atm, >0.6 atm, >0.7 atm, >0.8 atm, >0.9 atm, >1 atm, >1.1 atm, >1.2 atm, >1.3 atm, >1.4 atm, >1.5 atm, >1.6 atm, >1.7 atm, >1.8 atm, >1.9 atm, >2.0 atm, >2.1 atm, >2.2 atm, >2.3 atm, >2.4 atm, >2.5 atm, >2.6 atm, >2.7 atm, >2.8 atm, >2.9 atm, >3.0 atm, >3.5 atm, >4.0 atm, >4.5 atm, >5.0 atm, >5.5 atm, >6.0 atm, >6.5 atm, >7.0 atm, >7.5 atm, >8.0 atm, >8.5 atm, >9.0 atm, >10.0 atm, >11.0 atm, >12.0 atm, >13.0 atm, >14.0 atm, >15.0 atm, >16.0 atm, >17.0 atm, >18.0 atm, >19.0 atm or >20.0 atm.

In some embodiments, the GSHV in an OCM reaction catalyzed by the OCM catalytic materials is >5,000/hr, >10,000/hr, >15,000/hr, >20,000/hr, >50,000/hr, >75,000/hr, >100,000/hr, >120,000/hr, >130,000/hr, >150,000/hr, >200,000/hr, >250,000/hr, >300,000/hr, >350,000/hr, >400,000/hr, >450,000/hr, >500,000/hr, >750,000/hr, >1,000,000/hr, >2,000,000/hr, >3,000,000/hr, or >4,000,000/hr.

The OCM reactions catalyzed by the disclosed OCM catalytic materials can be performed (and still maintain high C₂₊ yield, C₂₊ selectivity, conversion, etc.) using O₂ sources other than pure O₂. For example, in some embodiments the O₂ source in an OCM reaction catalyzed by the disclosed OCM catalytic materials is air, oxygen enriched air, pure oxygen, oxygen diluted with nitrogen (or another inert gas) or oxygen diluted with CO₂. In certain embodiments, the O₂ source is O₂ diluted by >99%, >98%, >97%, >96%, >95%, >94%, >93%, >92%, >91%, >90%, >85%, >80%, >75%, >70%, >65%, >60%, >55%, >50%, >45%, >40%, >35%, >30%, >25%, >20%, >15%, >10%, >9%, >8%, >7%, >6%, >5%, >4%, >3%, >2% or >1% with CO₂ or an inert gas, for example nitrogen or helium.

The disclosed OCM catalytic materials are also very stable under conditions required to perform any number of catalytic reactions, for example the OCM reaction. The stability of the OCM catalytic materials is defined as the length of time a catalyst will maintain its catalytic performance without a significant decrease in performance (e.g., a decrease >20%, >15%, >10%, >5%, or greater than 1% in C₂₊ yield, C₂₊ selectivity or conversion, etc.). In some embodiments, the OCM catalytic materials have stability under conditions required for the OCM reaction of >1 hr, >5 hrs, >10 hrs, >20 hrs, >50 hrs, >80 hrs, >90 hrs, >100 hrs, >150 hrs, >200 hrs, >250 hrs, >300 hrs, >350 hrs, >400 hrs, >450 hrs, >500 hrs, >550 hrs, >600 hrs, >650 hrs, >700 hrs, >750 hrs, >800 hrs, >850 hrs, >900 hrs, >950 hrs, >1,000 hrs, >2,000 hrs, >3,000 hrs, >4,000 hrs, >5,000 hrs, >6,000 hrs, >7,000 hrs, >8,000 hrs, >9,000 hrs, >10,000 hrs, >11,000 hrs, >12,000 hrs, >13,000 hrs, >14,000 hrs, >15,000 hrs, >16,000 hrs, >17,000 hrs, >18,000 hrs, >19,000 hrs, >20,000 hrs, >1 yr, >2 yrs, >3 yrs, >4 yrs or >5 yrs.

In some embodiments, the ratio of ethylene to ethane in an OCM reaction catalyzed by the OCM catalytic materials is >0.3, >0.4, >0.5, >0.6, >0.7, >0.8, >0.9, >1, >1.1, >1.2, >1.3, >1.4, >1.5, >1.6, >1.7, >1.8, >1.9, >2.0, >2.1, >2.2, >2.3, >2.4, >2.5, >2.6, >2.7, >2.8, >2.9, >3.0, >3.5, >4.0, >4.5, >5.0, >5.5, >6.0, >6.5, >7.0, >7.5, >8.0, >8.5, >9.0, >9.5, >10.0.

In some embodiments, the conversion of methane in an OCM reaction catalyzed by the OCM catalytic materials is greater than 5%, greater than 10%, greater than 20%, greater than 30%, greater than 40%, greater than 50%, greater than 60%, greater than 75%, or greater than 90%.

In some embodiments, the C2+ selectivity in an OCM reaction catalyzed by the OCM catalytic materials is greater than 30%, greater than 35%, greater than 40%, greater than 45%, greater than 50%, greater than 55%, greater than 60%, greater than 65%, greater than 70%, or greater than 75%.

In some embodiments, the yield of ethylene in an OCM reaction catalyzed by the OCM catalytic materials is greater than 10%, greater than 20%, greater than 30%, greater than 50%, greater than 75%, or greater than 90%. In some other embodiments, the yield of C₂₊ hydrocarbons in an OCM reaction catalyzed by the OCM catalytic materials is greater than 5%, greater than 10%, greater than 20%, greater than 30%, greater than 50%, greater than 75%, or greater than 90%.

In various embodiments of the foregoing methods for the oxidative coupling of methane, a method for the oxidative coupling of methane to C₂₊ hydrocarbons under adiabatic conditions is provided, the method comprising passing a feed gas comprising methane at a linear velocity of 1 m/s or higher through a packed catalyst bed, the packed catalyst bed comprising any of the OCM catalytic materials described herein.

In any of the embodiments described herein, the linear velocity in an OCM method ranges from about 0.1 m/s to about 10 m/s, for example about 1 m/s to about 10 m/s or about 1 to about 5 m/s. In some embodiments, the linear velocity ranges from about 2 m/s to about 10 m/s, for examples from about 2 m/s to about 4 m/s.

In other embodiments, a C₂₊ selectivity for the conversion of methane to C₂₊ hydrocarbons is greater than about 50%, for example greater than about 55% or even greater than about 60%.

In some embodiments of the OCM methods described herein, the method produces ethylene which is employed as starting material to make downstream products of ethylene. In other embodiments of the OCM methods described herein, the final product is polymer-grade ethylene product (greater than 99 wt % ethylene, e.g. 99.96 wt % or greater).

In some embodiments, a method for the oxidative coupling of methane to C₂₊ hydrocarbons comprising injecting a feed gas comprising methane, oxygen and steam into a reactor section containing the disclosed OCM catalytic material. The feed gas contacts the disclosed OCM catalytic material to produce a product gas comprising C₂₊ hydrocarbons.

In such embodiments, the disclosed OCM material is capable of reaching a C₂₊ selectivity described above with a molar steam to methane ratio of above at least about 0.25:1, above at least about 0.5:1, above at least about 0.75:1, above at least about 1:1, above at least about 1.5:1, above at least about 2:1, above at least about 3:1, above at least about 4:1 or above at least about 5:1.

In such embodiments, the disclosed OCM catalytic material is capable of reaching a methane conversion described above with a molar steam to methane ratio of above at least about 0.25:1, above at least about 0.5:1, above at least about 0.75:1, above at least about 1:1, above at least about 1.5:1, above at least about 2:1, above at least about 3:1, above at least about 4:1 or above at least about 5:1.

In some embodiments, the disclosed OCM catalytic material can have improved performance characteristics for the OCM reaction in the presence of high temperature steam, while maintaining minimum physical strength properties required for commercial operation. In other embodiments, disclosed OCM catalytic material can maintain performance characteristics for the OCM reaction in the presence of high temperature steam at the same level when no steam is present in the feed gas, while maintaining minimum physical strength properties required for commercial operation. In still other embodiments, disclosed OCM catalytic material can have improved performance characteristics for the OCM reaction in the presence of high temperature steam, while decreasing other performance characteristics to a certain degree less than conventional catalysts.

In some embodiments, addition of steam to the feed gas can increase the methane conversion over an OCM catalyst by at least about 150%, at least about 200%, at least about 250%, at least about 300%, or at least about 400%.

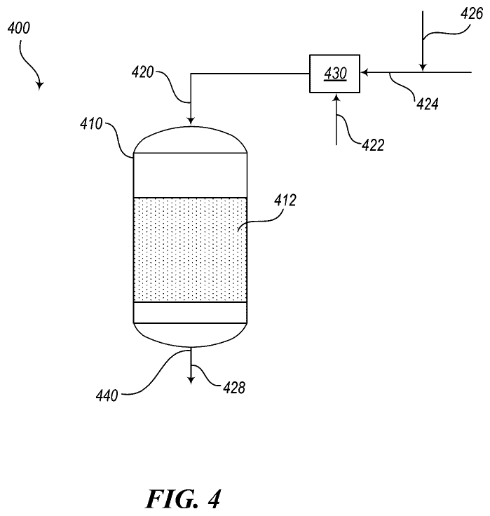

Referring to FIG. 4, a system 400 for performing an OCM reaction using an OCM catalytic material to produce C₂₊ compounds, including ethylene (C₂H₄), in accordance with the present disclosure is shown. In some embodiments, the system 400 includes an OCM reactor 410 comprising an OCM catalytic material 412 of the present disclosure. In some embodiments, the OCM catalytic material 412 may be layered in the OCM reactor 410. In some embodiments, the system 400 can include one or more OCM reactors 410 in series and/or parallel. In some embodiments, the OCM reactor 410 is an adiabatic reactor. In some embodiments, the OCM reactor 410 operates under adiabatic conditions. In some embodiments, the OCM reactor 410 is an isothermal reactor. In some embodiments, the OCM reactor 410 operates under isothermal conditions. The OCM reactor 410 may operate at a pressure of 0 kPa (gauge) to 2,000 kPa (gauge), including a pressure of 100 kPa (gauge) to 2,000 kPa (gauge), a pressure of 250 kPa (gauge) to 2,000 kPa (gauge), a pressure of 500 kPa (gauge) to 2,000 kPa (gauge), a pressure of 700 kPa (gauge) to 1,500 kPa (gauge), and also including a pressure of 750 kPa (gauge) to 1,250 kPa (gauge).

As shown in FIG. 4, a gas mixture feed 420 can enter the OCM reactor 410 via a feed inlet. The gas mixture feed 420 comprises methane (CH₄) and oxygen (O₂). In some embodiments, methane and oxygen are fed to the OCM reactor 410 via separate inlets. The gas mixture feed 420 may be formed by mixing together a hydrocarbon stream 422 and an oxidant stream 424 comprising oxygen. The reactants can be provided to the OCM reactor 410 such that the reactant mix in the OCM reactor 410 to form a reactant mixture prior to contacting the OCM catalytic material 412. In some embodiments, the oxidant stream 424 is provided by an air stream or an oxygen stream that is generated by an air separation unit. In some embodiments, the oxidant stream 424 further comprises steam, which may be injected or otherwise added into the oxidant stream 424 via line 426. In some embodiments, the hydrocarbon stream 422 is mixed with the oxidant stream 424 in a mixing device 430.

The feed temperature should be high enough to allow the OCM reaction to light-off. In some embodiments, the gas mixture feed 420 is preheated to a temperature from 450° C. to 800° C. When contacted by the heated gas mixture, the OCM catalytic material 412 becomes activated and initiates an OCM reaction to produce an OCM effluent 440 comprising C₂₊ compounds including C₂H₄ and C₂H₆ and non-C₂₊ impurities comprising one or more of CO, CH₄, H₂, and CO₂. Because the OCM reaction is exothermic, the temperature at which the OCM reaction is performed and/or maintained is typically higher than the temperature of the heated gas mixture used to activate or light-off the OCM catalyst. In some embodiments, the OCM reaction is performed and/or maintained at a temperature of 450° C. to 950° C., for example, a temperature of 500° C. to 950° C., 550° C. to 950° C., 600° C. to 950° C., 650° C. to 950° C., 700° C. to 950° C., 750° C. to 950° C., 800° C. to 950° C., 850° C. to 950° C., and also including a temperature of 875° C. to 925° C. In some embodiments, the OCM catalytic material 412 and the gas mixture feed 420 can be heated to approximately the same temperature. The OCM effluent 440 can exit the OCM reactor 410 via product outlet 428.

### Selective Oxidation Catalytic Materials

One of the primary obstacles to overcome in achieving a commercially viable OCM process has been the high ignition and reaction temperatures required to make the OCM reaction proceed. Conventional methods of performing an OCM reaction typically utilize a high temperature furnace to preheat the reactant feed(s) to the temperature required to ignite or “light-off” the OCM catalyst to initiate the OCM reaction. Operation of the high temperature furnace can be quite expensive and also creates a source of emissions, as the high temperature furnace burns fuel (e.g., natural gas) to generate the required heat.

Moreover, mixing the reactant feeds (i.e., methane-containing feed and oxygen-containing feed) at the high temperatures required to light-off the OCM catalyst and carry out the OCM reaction can create potential process safety and operation issues. For example, mixing the reactant feeds at high temperatures can cause ignition of the mixed reactant gas feed prior to reaching the OCM catalyst bed. Such premature ignition can cause damage to the reactor as well as the OCM catalyst bed. Damage to the catalyst can impede the ability of the catalyst to light-off and initiate the OCM reaction. Furthermore, premature ignition of the mixed reactant gas feed can reduce the selectivity of the OCM reaction due to the conversion of methane to carbon dioxide and carbon monoxide instead of the desired C₂₊ products. In addition, a wide variety of process upsets (e.g., flow upsets, temperature deviations) can lead to premature ignition of the mixed reactant gas feed. Accordingly, mixing the reactant feeds at the high temperatures required to light-off the OCM catalyst and carry out the OCM reaction may result in process safety issues.

In some embodiments, the OCM catalytic material may be combined with a selective oxidation catalytic material for performing an OCM reaction to produce C₂₊ compounds using a low temperature gas mixture feed. In some embodiments, the OCM reaction may be performed utilizing a gas mixture feed having a temperature of less than or equal to 375° C., for example, less than or equal to 350° C., less than or equal to 325° C., or less than or equal to 300° C., when a selective oxidation catalytic material is used in addition to the OCM catalytic material of the present disclosure. The selective oxidation catalytic material can help to improve the overall safety of the OCM process by avoiding issues associated with mixing methane and oxygen at elevated temperatures (e.g., 450° C. or more). In addition, the selective oxidation catalytic material can help to obviate the need for a high temperature furnace or preheater typically used in conventional OCM processes, which thereby eliminates the associated furnace/preheater air emissions from the process. Furthermore, the selective oxidation catalytic material can help to promote the C2+ selectivity of the OCM reaction by preferentially combusting hydrogen, carbon monoxide, or both over methane in the gas mixture feed.

In one aspect of the present disclosure, a catalytic material is provided for use in selective oxidation reaction of at least hydrogen (also referred to as “selective oxidation catalytic material”). By employing a selective oxidation catalytic material in combination with the OCM catalytic material described above, the present disclosure allows for performing an OCM reaction using a low temperature gas mixture feed.

In some embodiments, the selective oxidation catalytic material comprises a selective oxidation catalyst (SOC) on a support. In some embodiments, the support is a hibonite-type support comprising alkaline earth metal hexaaluminate described above. In some other embodiments, the support is silica, alumina, titania, zirconia, ceria, hafnia, cordierite, silicon carbide, aluminum hydroxide, monocalcium aluminate, tricalcium aluminate, and zeolites (e.g., ZSM-5 zeolite, Y zeolite, MCM-41 zeolite).

The SOC can be any catalyst composition that is capable of selectively or preferentially oxidizing hydrogen and/or CO over methane. In some embodiments, the SOC comprises at least one of a metal, a metal oxide, or a mixed metal oxide. Exemplary metals, metal oxides, and mixed metal oxides suitable for use as the SOC of the present disclosure include, but are not limited to, platinum, platinum oxide, chromium, chromium (II) oxide, chromium (III) oxide, chromium (VI) oxide, copper, copper (I) oxide, copper (II) oxide, copper (III) oxide, palladium, palladium (II) oxide, cobalt, cobalt (II) oxide, cobalt (III) oxide, iron, iron (II) oxide, iron (III) oxide, manganese, manganese (II) oxide, manganese (III) oxide, gold, gold (III) oxide, cerium, cerium (IV) oxide, tin, tin (II) oxide, tin (IV) oxide, bismuth, bismuth (III) oxide, indium, indium (III) oxide, molybdenum, molybdenum (IV) oxide, molybdenum (VI) oxide, antimony, antimony (III) oxide, lanthanum, lanthanum (III) oxide, aluminum, silver, osmium, tungsten, lead, zinc, nickel, rhodium, ruthenium, thallium, tellurium, germanium, gadolinium, Bi₂Mo₃O₁₂, In₂Mo₃O₁₂, Al₂Mo₃O₁₂, Fe₂Mo₃O₁₂, Cr₂Mo₃O₁₂, La₂Mo₃O₁₂, Ce₂Mo₃O₁₂, or combinations thereof.

In some embodiments, the SOC comprises at least one noble metal selected from the group consisting of silver (Ag), palladium (Pd), platinum (Pt), rhodium (Rh), ruthenium (Ru), gold (Au), iridium or osmium (Os), and combinations thereof. In some more specific embodiments, the SOC comprises palladium or platinum.

The amount of the SOC in the selective oxidation catalytic material can vary, for example, from about 0.001 wt % to 5 wt %, such as from about 0.001 wt % to about 0.005 wt %, from about 0.005 wt % to about 0.01 wt %, from about 0.01 wt % to about 0.05 wt %, from about 0.05 wt % to about 0.1 wt %, from about 0.1 wt % to about 0.5 wt %, from about 0.5 wt % to about 1 wt %, from about 1 wt % to about 2 wt %, from about 2 wt % to about 3 wt %, from about 3 wt % to about 4 wt %, or from about 4 wt % to about 5 wt %. In some embodiments, the SOC catalyst is present in an amount of about 0.001 wt %, about 0.002 wt %, about 0.005 wt %, about 0.008 wt %, about 0.01 wt %, about 0.02 wt %, about 0.05 wt %, about 0.1 wt %, about 0.2 wt %, about 0.5 wt %, about 1 wt %, about 1.5 wt %, about 2 wt %, about 2.5 wt %, about 3.0 wt %, about 3.5 wt %, about 4 wt %, or about 4.5 wt %, or about 5 wt %.

### Preparation of Selective Oxidation Catalytic Materials

In one aspect, the present disclosure provides a method for preparation of selective oxidation catalytic materials described above.

In some embodiments, the selective oxidation catalytic materials are prepared by dissolving a SOC metal salt in a solvent to provide a catalyst precursor solution. Examples of solvents include, but are not limited to, water, methanol, ethanol, propanol or isopropanol, butanol, tetrahydrofuran, diethyl ether, dimethoxyethane, acetonitrile, toluene, or mixtures thereof. In some embodiments, the SOC metal salt includes a metal chloride, a metal nitrate, a metal acetate, a metal citrate, a metal lactate, a metal carbonate, or combinations thereof. In some embodiments, the metal salt is a palladium acid. In some embodiments, the catalyst precursor solution is prepared by dissolving tetraammineplatinum nitrate (Pt(NH₃)₄(NO₃)₂) in DI water.

The catalyst precursor solution is applied to the support pellets to provide support pellets comprising the base catalyst precursor and the dopant precursor. In some embodiments, application of the catalyst precursor solution is performed by immersion of the support pellets in the catalyst precursor solution or by so-called “incipient wetness” impregnation where the volume of solution used equates approximately to the pore volume of the support pellets. Impregnation may be performed at ambient or elevated temperature and at atmospheric or elevated pressure. In some embodiments, the impregnation is performed at ambient temperature (i.e., 20 to 25° C.) and under atmospheric pressure (about 1 bar abs) for about one hour.

Following impregnation, the support pellets are removed from the catalyst precursor solution, and are then dried and calcined to provide the selective oxidation catalytic materials of the present disclosure. The drying temperatures may be from 80 to 120° C. and drying times may be from 1 minutes to 6 hours. In some embodiments, the support pellets are dried in a convection oven at 110° C. for about four hours. The oven temperature is increased from the ambient temperature to 110° C. at a rate of 5° C./min. In some embodiments, the support pellets are dried in stagnant air with no gas flow. After drying, the oven temperature is increased from the drying temperature, e.g., 110° C., to a calcination temperature at a rate of 5° C./min. The calcination temperatures may be from 400 to 900° C. such as 400° C., 500° C., 600° C., 700° C., 800° C., or 900° C., with calcination times ranging from 5 minutes to 5 hours. In some embodiments, the catalyst precursor-impregnated support pellets are calcined at 600° C. for about 4 hours to decompose the metal salts to metal elements.

After the calcination step, the support pellets comprising SOC are cooled from 600° C. to ambient temperature to make the supported SOC.

During the heating steps, the cooling steps, and the steps of maintaining the temperature, the support pellets may be exposed to an atmosphere comprising oxygen. The atmosphere may comprise 15 mole % to 25 mole % oxygen and 75 mole % to 85 mole % nitrogen. In some embodiments, the atmosphere may be air.

The impregnation step and calcination step could be repeated one or more times in order to increase the amount of SOC on the support pellets.

### Systems and Methods for Oxidative Coupling of Methane Using Selective Oxidation and OCM Catalytic Materials

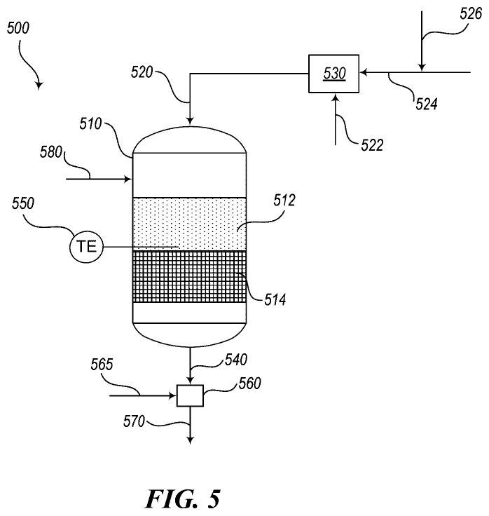

Referring now to FIG. 5, a system 500 for performing an OCM reaction using selective oxidation and OCM catalytic materials to produce C₂₊ compounds, including ethylene (C₂H₄), in accordance with the present disclosure is shown. The system 500 includes an OCM reactor 510 comprising a selective oxidation catalytic material 512 and an OCM catalytic material 514. In certain embodiments, the system 500 can include one or more OCM reactors 510 in series and/or parallel. In certain embodiments, the OCM reactor 510 is an adiabatic reactor. In some embodiments, the OCM reactor 510 operates under adiabatic conditions. In certain embodiments, the OCM reactor 510 is an isothermal reactor. In certain embodiments, the OCM reactor 510 operates under isothermal conditions. The OCM reactor 510 may operate at a pressure of 0 kPa (gauge) to 2,000 kPa (gauge), including a pressure of 100 kPa (gauge) to 2,000 kPa (gauge), a pressure of 250 kPa (gauge) to 2,000 kPa (gauge), a pressure of 500 kPa (gauge) to 2,000 kPa (gauge), a pressure of 700 kPa (gauge) to 1,500 kPa (gauge), and also including a pressure of 750 kPa (gauge) to 1,250 kPa (gauge).

As shown in FIG. 5, the OCM reactor 510 receives a gas mixture feed 520 at an inlet of the OCM reactor 510. The gas mixture feed 520 comprises methane (CH₄), oxygen (O₂), hydrogen (H₂), and carbon monoxide (CO). The gas mixture feed 520 may be formed by mixing together a hydrocarbon stream 522 comprising CH₄, H₂, and CO and an oxidant stream 524 comprising O₂. In some embodiments, the oxidant stream 524 is provided by an air stream or an O₂ stream that is generated by an air separation unit. In some embodiments, the oxidant stream 524 further comprises steam, which may be injected or otherwise added into the oxidant stream 524 via line 526. In some embodiments, the hydrocarbon stream 522 is mixed with the oxidant stream 524 in a mixing device 530. The mixing device 530 may be any known device suitable for mixing together a first gas stream comprising hydrocarbons and a second gas stream comprising oxygen such as, for example, the mixing device described in U.S. Pat. No. 3,706,534. In certain aspects, the mixing device 530 comprises process piping or a mixing tee.

In some embodiments, the gas mixture feed 520 is introduced in the OCM reactor 510 at a temperature of less than or equal to 375° C., for example, less than or equal to 350° C., less than or equal to 325° C. or less than or equal to 300° C. In some embodiments, the gas mixture feed 520 is introduced in the OCM reactor 510 at a pressure of 200 kPa (gauge) to 1,400 kPa (gauge), including a pressure of 500 kPa (gauge) to 1,200 kPa (gauge), 600 kPa (gauge) to 1,100 kPa (gauge), 700 kPa (gauge) to 1,000 kPa (gauge), and also including a pressure of 750 kPa (gauge) to 950 kPa (gauge). In some embodiments, the gas mixture feed 520 has a temperature ranging from 15° C. to 375° C. at the inlet of the OCM reactor 510, including a temperature ranging from 15° C. to 50° C., from 50° C. to 75° C., from 75° C. to 100° C., from 100° C. to 125° C., from 125° C. to 150° C., from 150° C. to 175° C., from 175° C. to 200° C., from 200° C. to 225° C., from 225° C. to 250° C., from 250° C. to 275° C., from 275° C. to 300° C., from 300° C. to 325° C., from 325° C. to 350° C., or from 350° C. to 375° C., at the inlet of the OCM reactor 510. In some embodiments, the temperature of the gas mixture feed 520 at the inlet of the OCM reactor 510 is from 15° C. to 250° C. In some embodiments, the hydrocarbon stream 522 used to form the gas mixture feed 520 has a temperature of less than or equal to 375° C., including a temperature ranging from 15° C. to 50° C., from 50° C. to 75° C., from 75° C. to 100° C., from 100° C. to 125° C., from 125° C. to 150° C., from 150° C. to 175° C., from 175° C. to 200° C., from 200° C. to 225° C., from 225° C. to 250° C., from 250° C. to 275° C., from 275° C. to 300° C., from 300° C. to 325° C., from 325° C. to 350° C., or from 350° C. to 375° C. In some embodiments, the oxidant stream 524 used to form the gas mixture feed 520 has a temperature ranging from 0° C. to 250° C., including a temperature ranging from 0° C. to 10° C., from 10° C. to 20° C., from 20° C. to 30° C., from 30° C. to 40° C., from 40° C. to 50° C., from 50° C. to 100° C., from 100° C. to 150° C., from 150° C. to 200° C., or from 200° C. to 250° C.

By providing the gas mixture feed 520 to the OCM reactor 510 at a relatively low temperature, embodiments of the present disclosure improve the safety of the OCM process by essentially eliminating the risk of the gas mixture feed 520 prematurely igniting. While beneficial, the low temperature of the gas mixture feed 520 creates an obstacle to successfully performing an OCM reaction, namely, achieving the minimum temperature required to light-off or activate an OCM catalytic material 514 to initiate an OCM reaction. Depending on the OCM catalytic material 514, a minimum temperature of at least 450° C. (e.g., 450° C. to 700° C.) and more typically 500° C. to 700° C. is required to achieve light-off and initiate an OCM reaction.

Accordingly, to overcome this obstacle, embodiments of the present disclosure utilize a selective oxidation catalytic material 512 to autothermally heat the gas mixture feed 520 within the OCM reactor 510. When contacted by the gas mixture feed 520, the selective oxidation catalytic material 512 promotes combustion of hydrogen in the gas mixture feed 520 to generate a heated gas mixture having a temperature of at least 450° C. In some embodiments, when contacted by the gas mixture feed 520, the selective oxidation catalytic material 512 promotes combustion of hydrogen in the gas mixture feed 520 to generate a heated gas mixture having a temperature ranging from 450° C. to 950° C., including a temperature ranging from 450° C. to 500° C., from 500° C. to 550° C., from 550° C. to 600° C., from 600° C. to 650° C., from 650° C. to 700° C., from 700° C. to 750° C., from 750° C. to 800° C., from 800° C. to 850° C., from 850° C. to 900° C., or from 900° C. to 950° C. In some embodiments, the heated gas mixture has a temperature ranging from 500° C. to 800° C. Accordingly, the selective oxidation catalytic material 512 can be used to create a temperature difference between the temperature of the gas mixture feed 520 at the inlet of the OCM reactor 510 and the temperature of the heated gas mixture. In some embodiments, the temperature difference ranges from 75° C. to 600° C., including a temperature difference ranging from 75° C. to 100° C., from 100° C. to 150° C., from 150° C. to 200° C., from 200° C. to 250° C., from 250° C. to 300° C., from 300° C. to 350° C., from 350° C. to 400° C., from 400° C. to 450° C., from 450° C. to 500° C., from 500° C. to 550° C., or from 550° C. to 600° C.

By using a selective oxidation catalytic material 512, less CH₄ in the gas mixture feed 520 is consumed or combusted when autothermally raising the temperature of the gas mixture feed 520, which results in more CH₄ being available to participate in the OCM reaction. As a result, the selectivity of the OCM reaction for converting CH₄ to C₂₊ compounds (i.e., C₂₊ selectivity) can be maintained or improved. In some embodiments, the OCM reaction has a C₂₊ selectivity of at least 35%, at least 40%, at least 45%, at least 50%, at least 55%, at least 60%, at least 65%, or at least 70%. In some embodiments, the OCM reaction has a C₂₊ selectivity of 35% to 85%, including a C₂₊ selectivity of 40% to 85%, 50% to 85%, 60% to 85%, and also including a C₂₊ selectivity of 70% to 85%.

In addition to the selective oxidation catalytic material 512, the OCM reactor 510 also includes an OCM catalytic material 514 of the present disclosure to facilitate an OCM reaction to produce an OCM effluent 540 comprising C₂₊ compounds including C₂H₄ and C₂H₆ and non-C₂₊ impurities comprising one or more of CO, CH₄, H₂, and carbon dioxide (CO₂). When contacted by the heated gas mixture, the OCM catalytic material 514 becomes activated (i.e., achieves light-off) and initiates an OCM reaction to produce the OCM effluent 540 comprising C₂₊ compounds including C₂H₄ and C₂H₆ and non-C₂₊ impurities comprising one or more of CO, CH₄, H₂, and CO₂. Because the OCM reaction is exothermic, the temperature at which the OCM reaction is performed and/or maintained is typically higher than the temperature of the heated gas mixture used to activate or light-off the OCM catalytic material 514. In some embodiments, the OCM reaction is performed and/or maintained at a temperature ranging from 450° C. to 950° C., including a temperature ranging from 500° C. to 950° C., from 550° C. to 950° C., from 600° C. to 950° C., from 650° C. to 950° C., from 700° C. to 950° C., from 750° C. to 950° C., from 800° C. to 950° C., from 850° C. to 950° C., and also including a temperature ranging from 875° C. to 925° C. The OCM effluent 540 exiting the OCM reactor 510 will generally have a temperature corresponding to that of the OCM reaction temperature (i.e., 450° C. to 950° C.) and can be directed to downstream units and/or a separations subsystem for additional processing as described herein.

The selective oxidation catalytic material 512 and the OCM catalytic material 514 can be arranged within the OCM reactor 510 in a variety of ways. For example, in some embodiments, the OCM reactor 510 can include a first catalyst bed containing the selective oxidation catalytic material 512 and a second catalyst bed containing the OCM catalytic material 514 downstream of the first catalyst bed, as illustrated in FIG. 5. In some embodiments, the OCM reactor 510 can include a single catalyst bed comprising at least one layer of the selective oxidation catalytic material 512 and at least one layer of the OCM catalytic material 514. In some embodiments, the OCM reactor 510 can include a single catalyst bed that comprises a mixture of the selective oxidation catalytic material 512 and the OCM catalytic material 514. The OCM reactor 510 can also include one or more layers or beds of inert material (not shown), which may function as a physical support for the catalytic materials 512, 514 in a catalyst bed. Exemplary inert materials include, but are not limited to, silica, alumina, and zirconia. In some embodiments, the OCM reactor 510 can include a first catalyst bed containing the selective oxidation catalytic material 512 and an inert material, either as one or more distinct layers or as a mixture, and a second catalyst bed containing the OCM catalytic material 514.

With continued reference to FIG. 5, the OCM reactor 510 may comprise one or more temperature elements 550 to provide an indication of temperature within the OCM reactor 510. The temperature element 550 may be any suitable temperature measuring device including, but not limited to, a thermocouple and a resistance temperature detector (RTD). As seen in FIG. 5, a temperature element 550 is located in the selective oxidation catalytic material 512 upstream of the OCM catalytic material 514. For example, where the OCM reactor 510 includes a first catalyst bed comprising a selective oxidation catalytic material 512 and a second catalyst bed comprising an OCM catalytic material 514, the temperature element 550 is located proximate an end of the first catalyst bed and upstream of the second catalyst bed. In another example, where the OCM reactor 510 includes a single catalyst bed comprising a layer of selective oxidation catalytic material 512 upstream of a layer of OCM catalytic material 514, the temperature element 550 is located in the layer of selective oxidation catalytic material 512 proximate an interface between the layer of selective oxidation catalytic material 512 and the layer of OCM catalytic material 514. The temperature element 550 can be used to provide one or more signals indicative of a temperature of the heated gas mixture prior to the heated gas mixture contacting the OCM catalytic material 514. The temperature element 550 can be part of a control system operative to maintain the temperature of the heated gas mixture at a desired setpoint (e.g., the minimum OCM catalyst light-off temperature) by, for example, controlling or otherwise adjusting a temperature of the hydrocarbon stream 522, the oxidant stream 524, or both.

In some embodiments, embodiments of the present disclosure include a post-bed cracking (PBC) unit 560 for generating olefins (e.g., C₂H₄) from alkanes (e.g., C₂H₆, C₃H₈). The PBC unit 560 can be disposed downstream of the OCM reactor 510, as illustrated in FIG. 5. The PBC unit 560 may be a separate reactor, or the PBC unit 560 may be included as a section of the OCM reactor 510 (e.g., a section downstream of the OCM catalytic material 514 in the same vessel). As the OCM reaction is exothermic and generates heat, the heat generated by the OCM reaction can be used to crack alkanes (e.g., C₂H₆) to olefins (e.g., C₂H₄). The PBC unit 560 may perform the cracking at a temperature ranging from 600° C. to 1,000° C., including a temperature ranging from 700° C. to 1,000° C., from 750° C. to 1,000° C., from 775° C. to 1,000° C., and also including a temperature ranging from 800° C. to 950° C.

The PBC unit 560 can be used to crack additional external alkanes 565 (e.g., C₂H₆, C₃H₈) beyond those contained in the OCM effluent 540. The heat capacity in the OCM effluent 540 can be sufficient to crack some amount of additional external alkanes 565. The additional external alkanes 565 can be provided from a recycle stream of the process or an entirely separate source of alkanes. The external alkanes 565 can be heated prior to injection into the PBC unit 560. The external alkanes 565 can be heated by, for example, heat exchange with the OCM reactor 510 and/or the OCM effluent 540, or another process stream. A PBC effluent 570 exits the PBC unit 560 and includes a greater concentration of olefins (e.g., C₂H₄) and H₂ as compared to the OCM effluent 540.

In some embodiments, embodiments of the present disclosure include injecting an ignition component 580 into the OCM reactor 510. The ignition component 580 can be any substance that has a lower autoignition temperature than CH₄ such as, for example, dimethyl ether or methanol. The ignition component 580 can be provided to the OCM reactor 510 as an additional means (i.e., via combustion of the ignition component 580) to increase the temperature of the gas mixture feed 520. Although FIG. 5 illustrates the ignition component 580 being injected directly into the OCM reactor 510, the ignition component 580 could also be added to one or more of the gas mixture feed 520, the hydrocarbon stream 522, or the oxidant stream 524.

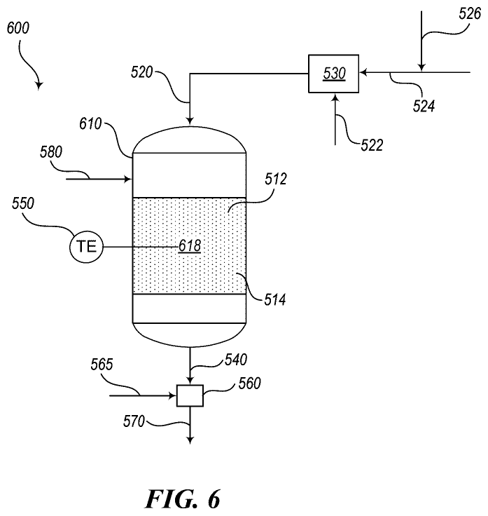

Referring now to FIG. 6, a system 600 for performing an OCM reaction using selective oxidation and OCM catalytic materials to produce C₂₊ compounds, including ethylene (C₂H₄), in accordance with the present disclosure is shown. The system 600 illustrated in FIG. 6 is similar to the systems 500 depicted in FIG. 5 and like numerals are used in FIG. 6 to identify like components. The primary difference between the system 600 illustrated in FIG. 6 and the system 500 illustrated in FIG. 5 is that the OCM reactor 610 includes a single catalyst bed comprising a selective oxidation catalytic material and an OCM catalytic material.

As shown in FIG. 6, the OCM reactor 610 includes a single catalyst bed 618 that comprises a selective oxidation catalytic material 512 and an OCM catalytic material 514. In some embodiments, the catalyst bed 618 can comprise at least one layer of the selective oxidation catalytic material 512 and at least one layer of the OCM catalytic material 514. In some embodiments, the catalyst bed 618 can comprise a mixture of the selective oxidation catalytic material 512 and the OCM catalytic material 514.

### Downstream Products

As noted above, the catalytic materials disclosed herein are useful in reactions for the preparation of a number of valuable hydrocarbon compounds. For example, in one embodiment the catalytic materials are useful for the preparation of ethylene from methane via the OCM reaction. Ethylene can be converted into many various compounds including low density polyethylene, high density polyethylene, ethylene dichloride, ethylene oxide, ethylbenzene, linear alcohols, vinyl acetate, alkanes, alpha olefins, various hydrocarbon-based fuels, ethanol, and the like. These compounds can then be further processed using methods well known to one of ordinary skill in the art to obtain other valuable chemicals and consumer products. Propylene can be analogously converted into various compounds and consumer goods including polypropylenes, propylene oxides, propanol, and the like.

Accordingly, in some embodiments, the present disclosure provides a method of preparing downstream products of ethylene. The method comprises converting ethylene into a downstream product of ethylene by oligomerization of ethylene, wherein the ethylene has been prepared via an OCM reaction employing OCM catalytic materials disclosed herein alone or in combination with SOC materials disclosed herein. In some embodiments, the downstream product of ethylene is low density polyethylene, high density polyethylene, ethylene dichloride, ethylene oxide, ethylbenzene, ethanol, or vinyl acetate. In other embodiments, the downstream product of ethylene is a hydrocarbon fuel such as natural gasoline or a C₄-C₁₄ hydrocarbon, including alkanes, alkenes and aromatics. Some specific examples include 1-butene, 1-hexene, 1-octene, hexane, octane, benzene, toluene, xylenes, and the like.

Oligomerization of ethylene to higher hydrocarbons (e.g., C₄-C₁₄) may be effected by use of any number of catalysts known to those skilled in the art. Examples of such catalysts include catalytic zeolites, crystalline borosilicate molecular sieves, homogeneous metal halide catalysts, Cr catalysts with pyrrole ligands or other catalysts. Exemplary methods for the conversion of ethylene into higher hydrocarbon products are disclosed in the following references: Catalysis Science & Technology (2011), 1 (1), 69-75; Coordination Chemistry Reviews (2011), 255 (7-8), 861-880; Eur. Pat. Appl. (2011), EP 2287142 A1 20110223; Organometallics (2011), 30 (5), 935-941; Designed Monomers and Polymers (2011), 14 (1), 1-23; Journal of Organometallic Chemistry 689 (2004) 3641-3668; Chemistry—A European Journal (2010), 16 (26), 7670-7676; Acc. Chem. Res. 2005, 38, 784-793; Journal of Organometallic Chemistry, 695 (10-11): 1541-1549 May 15 2010; Catalysis Today Volume 6, Issue 3, January 1990, Pages 329-349; U.S. Pat. Nos. 5,968,866; 6,800,702; 6,521,806; 7,829,749; 7,867,938; 7,910,670; 7,414,006 and Chem. Commun., 2002, 858-859, each of which are hereby incorporated in their entirety by reference.

The following examples are provided for purposes of illustration, not limitation.

### EXAMPLES

### Example 1

### Preparation of Hibonite Supported SrO/Nd₂O₃ OCM Catalyst

To prepare this OCM catalytic material comprising SrO doped Nd₂O₃ on a hibonite support with 15.0 wt % Nd₂O₃-0.5 wt % SrO, surface areas of the hibonite pellets were measured by BET (Brunauer, Emmett, Teller) measurements and pore volumes were measured by Mercury Intrusion Porosimetry (MIP). The average pore volume of the hibonite pellets was calculated and an amount of a catalyst precursor solution that can be absorbed by the hibonite pellets was estimated. Next, the catalyst precursor solution was prepared at room temperature by dissolving neodymium (II) nitrate hexahydrate (177.8 g) and strontium nitrate (4.19 g) in deionized (DI) water (100 ml). The catalyst precursor solution was stirred until neodymium (II) nitrate hexahydrate and strontium nitrate were completely dissolved. 42.28 g of hibonite pellets were placed in a steel wire basket, and the basket was immersed in the catalyst precursor solution and soaked for one hour so that about 11 ml of the catalyst precursor solution was absorbed by the hibonite pellets, The soaked hibonite pellets were then transferred from the basket and put in a ceramic crucible and dried in a convection oven by increasing the temperature of the convection oven from ambient to 110° C. at a ramping rate of 5° C./min and holding at 110° C. for 4 hours in stagnant air (no gas flow). Subsequently, the temperature of the convection oven was increased from 110° C. to 800° C. at a ramping rate of 5° C./min. The soaked hibonite pellets were calcined in the convection oven by being hold at 800° C. for 5 hours, and then cooled to room temperature at a rate of 20° C./min. The SrO/Nd₂O₃ impregnated hibonite pellets were ground and sieved to have a particle size range of 200 μm to 500 μm.

### Example 2

### Hydrothermal Stability of Hibonite Supported SrO/Nd₂O₃ OCM Catalyst

The hydrothermal stability of the hibonite supported SrO/Nd₂O₃ OCM catalyst of Example 1 was tested in a 2 mm reactor. After loading the hibonite supported OCM catalytic material into the reactor, the reactor was heated to 700° C. A gas mixture feed consisting of about 63 mol % CH₄, 7.1 mol % O₂, and 25 mol % H₂O, balanced with He was flowed over the catalyst bed at a space velocity (GHSV) of 6167 h⁻¹ and a pressure of 8 barg.

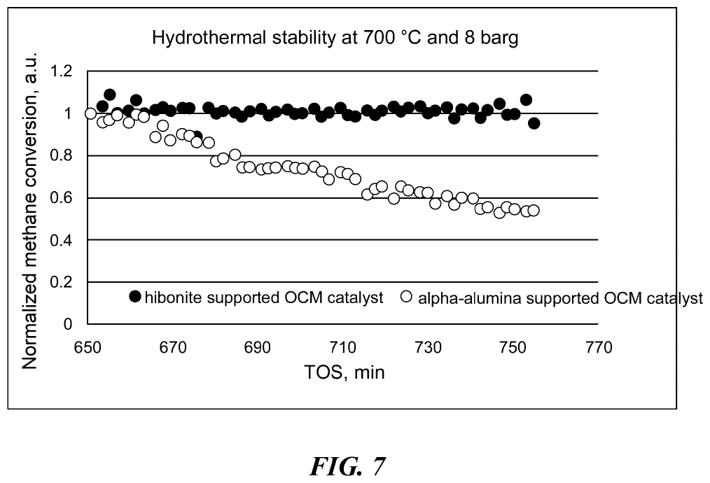

FIG. 7 shows the comparative results in hydrothermal stability for the hibonite-supported OCM catalyst and an alpha-alumina supported OCM catalyst as a function of time on stream (TOS). As shown in FIG. 7, the hibonite-supported OCM catalyst of the present disclosure has a higher hydrothermal stability than the alpha-alumina supported OCM catalyst. The methane conversion efficiency is decreased rapidly for the alpha-alumina supported OCM catalyst after the catalyst was exposed to the gas mixture feed for 670 minutes. In contrast, the methane conversion efficiency for the hibonite-support OCM catalyst remains the same after the catalyst was exposed to the gas mixture feed for 770 minutes.

### Example 3

### Preparation of 0.2 wt % Pt-Hibonite Selective Oxidation Catalytic Material

To prepare this selective oxidation catalytic material comprising Pt on a hibonite support at a level of 0.2 wt %, surface areas of the hibonite pellets were measured by BET (Brunauer, Emmett, Teller) measurements and pore volumes were measured by Mercury Intrusion Porosimetry (MIP). The average pore volume of the hibonite pellets was calculated and an amount of a catalyst precursor solution that can be absorbed by the hibonite pellets was estimated. Next, the catalyst precursor solution was prepared by dissolving hexachloroplatinic acid (3.18 g) in DI water (150 ml). The catalyst precursor solution was stirred until the hexachloroplatinic acid was completely dissolved. Hibonite pellets (199.6 g) were placed in a steel wire basket, and the basket was immersed in the catalyst precursor solution and soaked for one hour so that about 51 ml of the catalyst solution was absorbed by the hibonite pellets. The doped hibonite pellets were then transferred from the basket and put in a ceramic crucible. The doped hibonite pellets were dried in a convection oven by increasing the temperature of the convection oven from ambient to 110° C. at a ramping rate of 5° C./min and holding at 110° C. for 4 hours in stagnant air (no gas flow). Subsequently, the temperature of the convection oven was increased from 110° C. to 600° C. at a ramping rate of 5° C./min. The soaked hibonite pellets were calcined in the convection oven by being hold at 600° C. for 5 hours, and then cooled to room temperature at a rate of 20° C./min. The Pt impregnated hibonite pellets were ground and sieved to have a particle size range of 200 μm to 500 μm.

### Example 4

### Preparation of 0.1 wt % Pt-Hibonite Selective Oxidation Catalytic Material

To prepare this selective oxidation catalytic material comprising Pt on a hibonite support at a level of 0.1 wt %, surface areas of the hibonite pellets were measured by BET (Brunauer, Emmett, Teller) measurements and pore volumes were measured by Mercury Intrusion Porosimetry (MIP). The average pore volume of the hibonite pellets was calculated and an amount of a catalyst precursor solution that can be absorbed by the hibonite pellets was estimated. Next, the catalyst precursor solution was prepared by dissolving hexachloroplatinic acid (3.18 g) in DI water (150 ml). The catalyst precursor solution was stirred until the hexachloroplatinic acid was completely dissolved. Hibonite pellets (199.8 g) were placed in a steel wire basket, and the basket was immersed in the catalyst precursor solution and soaked for one hour so that about 51 ml of the catalyst solution was absorbed by the hibonite pellets. The soaked hibonite pellets were then transferred from the basket and put in a ceramic crucible. The soaked hibonite pellets were dried in a convection oven by increasing the temperature of the convection oven from ambient to 110° C. at a ramping rate of 5° C./min and holding at 110° C. for 4 hours in stagnant air (no gas flow). Subsequently, the temperature of the convection oven was increased from 110° C. to 600° C. at a ramping rate of 5° C./min. The soaked hibonite pellets were calcined in the convection oven by being hold at 600° C. for 5 hours, and then cooled to room temperature at a rate of 20° C./min. The Pt impregnated hibonite pellets were ground and sieved to have a particle size range of 200 μm to 500 μm.

### Example 5

### Effect of Catalyst Supports on Combustion Selectivity

The effect of catalyst supports on combustion selectivity was evaluated using a hibonite supported Pt SOC and a comparative gamma-alumina supported Pt SOC. The reactor was heated to 250° C. during the hydrogen combustion reaction. A gas mixture feed consisting of about 73.3 mol % CH₄, 12.1 mol % O₂, 4.5 mol % H₂, and 3.7 mol % CO, balanced with He was flowed over the catalyst bed at a space velocity (GHSV) of 37,400 h⁻¹ and a pressure of 8 barg.

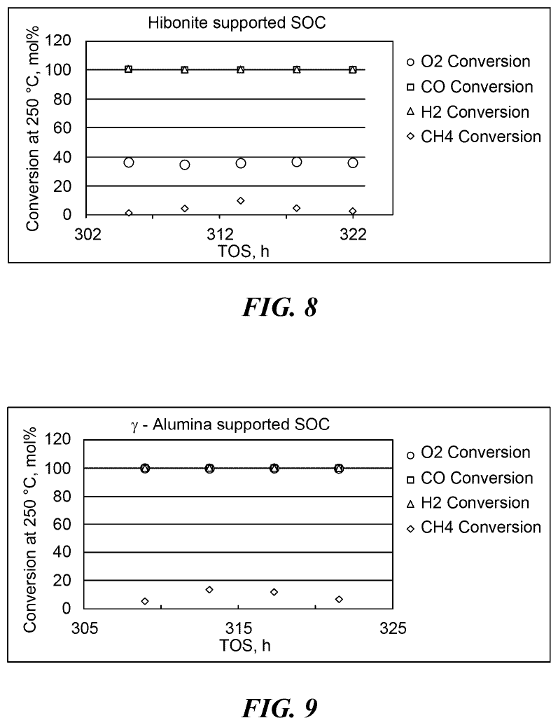

FIG. 8 shows the combustion selectivity of the hibonite supported Pt SOC prepared according to Example 3. FIG. 8 shows the combustion selectivity of the gamma-alumina supported Pd SOC. As shown in FIG. 8, the hibonite supported Pt SOC can selectively oxidize CO and H₂ over CH₄. In contrast, as shown in FIG. 9, the gamma-alumina supported Pt SOC also consumes O₂ during the SOC reaction, which reduces the SOC reaction efficiency.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet, including U.S. Provisional Patent Application No. 63/504,577, filed May 26, 2023, are incorporated herein by reference, in their entirety. Aspects of the embodiments can be modified, if necessary to employ concepts of the various patents, applications and publications to provide yet further embodiments. These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A catalytic material for oxidative coupling of methane (OCM), comprising:
a support comprising an alkaline earth metal hexaaluminate; and
an OCM catalyst in contact with the support, wherein the OCM catalyst comprises a rare earth oxide and at least one dopant.

2.-15. (canceled)

16. A catalytic material for selective oxidation of at least hydrogen over methane, comprising:
a support comprising an alkaline earth metal hexaaluminate; and
a selective oxidation catalyst (SOC) in contact with the support, wherein the SOC comprises a noble metal.

17.-28. (canceled)

29. A method for oxidative coupling of methane (OCM), comprising:
introducing a gas mixture feed comprising methane, oxygen and hydrogen to a reactor, wherein the reactor comprises a selective oxidation catalytic material and an OCM catalytic material;
contacting the gas mixture feed with the selective oxidation catalytic material to combust hydrogen in the gas mixture feed and generate a heated gas mixture having a temperature capable of initiating an OCM reaction, the selective oxidation catalytic material comprising a selective oxidation catalyst (SOC) in contact with a first support; and
contacting the heated gas mixture with the OCM catalytic material to initiate an OCM reaction and produce an OCM effluent comprising C₂₊ compounds including ethylene, the OCM catalytic material comprising an OCM catalyst in contact with a second support,
wherein at least one of the first support and the second support comprises an alkaline earth metal hexaaluminate.

30. The method of claim 29, wherein the OCM catalyst comprises a rare earth oxide and at least one dopant.

31. The method of claim 30, wherein the rare earth oxide comprises La₂O₃, CeO₂, Ce₂O₃, Pr₂O₃, Nd₂O₃, Sm₂O₃, Eu₂O₃, Gd₂O₃, Yb₂O₃, Y₂O₃, La₃NdO₆, LaNd₃O₆, La_{1.5}Nd_{2.5}O₆, La_{3.2}Nd_{1.5}O₆, La_{3.5}Nd_{0.5}O₆, La_{3.5}Nd_{0.5}O₆, La_{3.8}Nd_{0.2}O₆, LaYO₃, La₂Zr₂O₇ or La₂Ce₂O₇.

32.-33. (canceled)

34. The method of claim 30, wherein the dopant comprises an alkaline earth metal oxide selected from MgO, CaO, SrO and BaO or an alkaline earth metal element selected from Mg, Ca, Sr and Ba.

35.-36. (canceled)

37. The method of claim 29, wherein at least one of the first support and the second support comprises an alkaline earth metal hexaaluminate selected from calcium hexaaluminate (CaAl₁₂O₁₉), barium hexaaluminate (BaAl₁₂O₁₉), strontium hexaaluminate (SrAl₁₂O₁₉) and combinations thereof.

38.-40. (canceled)

41. The method of claim 29, wherein the first support and the second support are independently in the form of extruded or pelletized cylinders, spheres, rods, trilobes, tetralobes, rings, doughnuts, stars, cartwheels or strands.

42. (canceled)

43. The method of claim 29, wherein the first support and the second support independently comprise one or more holes extending therethrough.

44. The method of claim 29, wherein the SOC comprises a noble metal, wherein the noble metal comprises silver, palladium, platinum, rhodium, ruthenium, gold, iridium or osmium.

45. (canceled)

46. The method of claim 29, wherein the SOC comprises a metal, a metal oxide, or a mixed metal oxide comprising at least one of platinum, chromium, copper, palladium, cobalt, iron, manganese, gold, cerium, bismuth, indium, molybdenum, rhodium, ruthenium, germanium, gadolinium, antimony, thallium, tellurium, lead, zinc or tin.

47. The method of claim 29, wherein the OCM catalyst is present in the OCM catalytic material in an amount from 1 wt % to 25 wt %, and wherein the SOC is present in the selective oxidation catalytic material in an amount ranging from 0.001 wt % to 5 wt %.

48. The method of claim 29, wherein the gas mixture feed further comprises carbon monoxide, wherein the selective oxidation catalytic material preferentially combusts hydrogen and carbon monoxide over methane.

49.-50. (canceled)

51. The method of claim 29, wherein the gas mixture feed has a temperature ranging from 300° C. to 375° C. or from 15° C. to 250° C. at an inlet of the reactor.

52.-53. (canceled)

54. The method of claim 29, wherein the heated gas mixture feed has a temperature of at least 450° C.

55. The method of claim 54, wherein the heated gas mixture has a temperature ranging from 500° C. to 800° C.

56. The method of claim 29, wherein the OCM reaction is performed at a temperature ranging from 450° C. to 950° C.

57.-59. (canceled)

60. The method of claim 29, wherein the reactor comprises a first catalyst bed where the selective oxidation catalytic material is placed, and a second catalyst bed where the OCM catalytic material is placed, wherein the second catalyst bed is downstream of the first catalyst bed.

61. The method of claim 29, wherein the reactor comprises a single catalyst bed comprising a mixture of the selective oxidation catalytic material and the OCM catalytic material.

62. The method of claim 29, wherein the reactor comprises a single catalyst bed comprising at least one layer of the selective oxidation catalytic material and a least one layer of the OCM catalytic material.

63. A method for preparing a downstream product of ethylene, the method comprising converting ethylene into a downstream product of ethylene, wherein the ethylene has been prepared by the method of claim 29.

64.-67. (canceled)
